(19) United States
(12) Patent Application Publication
(10) Pub. No.: US 2024/0065665 A1
(43) Pub. Date: Feb. 29, 2024

(54) AUSCULTATORY SOUND ANALYSIS SYSTEM

(71) Applicant: YANCHERS CORPORATION, Kyoto-shi, Kyoto (JP)

(72) Inventor: Junichi SHIMADA, Kyoto (JP)

(73) Assignee: YANCHERS CORPORATION, Kyoto-shi, Kyoto (JP)

(21) Appl. No.: 18/270,105

(22) PCT Filed: Dec. 20, 2021
(86) PCT No.: PCT/JP2021/049035

§ 371 Date: Jun. 28, 2023

(30) Foreign Application Priority Data
Dec. 31, 2020 (JP) 2020-220113

(51) Int. Cl. A61B 7/04; A61B 5/00; A61B 5/0205

(52) U.S. Cl. CPC A61B 7/04; A61B 5/02055; A61B 5/742; A61B 5/01

(57) ABSTRACT

An auscultatory sound analysis system that makes it possible to chronologically understand qualitative changes in an auscultatory sound from an auscultation device that contributes to telemedicine avoiding medical practice involving users' close contact, in medical care having a possibility of a contact infection such as COVID-19. The auscultatory sound analysis system converts the auscultatory sound into digital data, further performing a spectrogram conversion, and chronologically outputting, along a time axis, strengths of a signal component at a specific frequency or in a specific frequency range. The system thereby makes it possible to output, to transfer data of, and to display quantitative and temporal changes in high-pitch crepitations caused by interstitial pneumonia from COVID-19 and to thus understand a development status of the disease.

### TECHNICAL FIELD

The present invention is related to an auscultatory sound analysis system, an output method thereof, and a program thereof for the purpose of understanding chronological changes in the “strength of a signal component” of a sound. The present invention is related to the auscultatory sound analysis system, the output method, a display method, and the program characterized by converting an auscultatory sound into a spectrogram and outputting strengths of the signal component at a specific frequency or in a specific frequency range along a time axis.

### BACKGROUND ART

Understanding states of a living body by using sounds originating from the living body is a common practice exercised as medical practice. For example, during an examination of internal medicine or the like, various types of organ sounds such as respiratory sounds, heart sounds, and others are listened to by using a stethoscope, so as to use results in diagnosing respiratory diseases, heart diseases, digestive diseases, and the like.

During a medical consultation, acquiring biological information by using a stethoscope is to acquire information at that particular moment in a real-time manner.

Conventionally, stethoscopes have primarily been of analog types. In recent years, however, a number of companies have developed electronic stethoscopes using digital technology. Certain stethoscopes are equipped with functions to adjust sound volume, to adjust frequency characteristics (for respiratory use, for heart sound use, etc.), and the like and have also become more user friendly. In addition, an electronic stethoscope keeping telemedicine in mind has also been developed (see WO2019067880 A1).

In recent years, data conversion has become easier, as central processing units of computers are provided with higher functionalities. A spectrogram denotes a result of putting a complex signal through a window function so as to calculate a frequency spectrum. A spectrogram can be expressed with a three-dimensional graph (time, frequencies, and strengths of a signal component). Spectrograms are used for analyzing a voiceprint, analyzing an animal sound, music, SONAR/RADAR, and audio processing. Spectrograms may be referred to as voiceprints. A device that generates a spectrogram is called a sonograph.

Further, an attempt has been made to output a result of visualizing a sound from a living body (an examined subject), in addition to simply outputting a sound originating from the living body, so as to acquire information visually for diagnosing purposes, while aiming at improvements of visibility, operability, and monitored elements (see Patent Literature 1 (Japanese Patent No. 3625294)).

By using a visual stethoscope disclosed in Patent Literature 1 referenced above, it is possible to display an auscultatory sound as three-dimensional information having frequencies, time, and amplitude information. It is therefore possible to output sounds, which is information that tends to rely on a subjective judgment, as objective information.

However, the visual stethoscope disclosed in Patent Literature 1 referenced above has problems described below.

Generally speaking, examiners using a stethoscope for diagnosing purposes make a judgment about suspected diseases, on the basis of sound information acquired through their ears and comparison with past experiences. With various types of diseases, auscultatory sounds each have a frequency characteristic. As an auscultatory sound at a normal time is compared with an auscultatory sound at the time of an illness, there is a difference in strength among signal components at different frequencies. Examiners make a judgment, by associating a frequency characteristic of auscultatory sound information acquired through their ears with their own experiences. However, it is not that diagnoses are made by bringing diseases into association with specific frequencies of auscultatory sounds, like an auscultatory sound caused by one of various diseases specifically having a frequency of how many Hz corresponds to specific disease A, while an auscultatory sound having another frequency (Hz) corresponds to another disease B. Accordingly, even when the frequencies, the time, and the amplitude information of an auscultatory sound are visualized, examiners need to be specially trained in order for the examiners to be able to make a diagnosis on the basis of the visual information of the auscultatory sound. It would not be easy for examiners to go through such new training during their busy schedule while diagnosing patients in their clinical environments.

### CITATION LIST

### Patent Literature

Patent Literature 1:) Japanese Patent No. 3625294

In Patent Literature 1 referenced above, a spectrogram of an auscultatory sound in a short time span is acquired in a “real-time” manner, while conventional medical consultations are indeed taken into account. Being an anesthesiologist, the inventor of the patent conceived of the technical idea that contributes to three-dimensional, visual, and objective diagnosing processes based on “respiratory sounds and heart sounds at those particular moments in a real-time manner”.

However, the data of the auscultatory sound itself is “real-time” data from that particular medical practice, no matter how much care is taken to acquire the auscultatory sound in a “real-time” manner, by acquiring the auscultatory sound from a patient and acquiring the auscultatory sound at that moment in the “real-time” manner, so as to be three-dimensionally turned into an image and to be finely tuned to avoid individual differences among users' auditory characteristics. Reference Document 1: Japanese Patent Laid-Open No. 2012-223509 A.

For these reasons, the devices hitherto known merely provide means for electrically transmitting an auscultatory sound as sound data to a remote location and means for objectively understanding a real-time auscultatory sound visually.

### SUMMARY OF INVENTION

### Technical Problem

Regarding the novel coronavirus disease (hereinafter, “COVID-19”), among the PCR positive patients, it is estimated that as high as nearly 80% of the positive patients such as those who had no or mild symptoms were forced to wait and watch without going to medical institutions.

Regarding COVID-19, however, there has been a problem where, among the PCR positive patients, some patients who had no or mild symptoms and were forced to wait and watch without going to medical institutions had a sudden change in their viral interstitial pneumonia condition and died.

Regarding COVID-19, among the PCR positive patients, even in the cases where some patients who had no or mild symptoms and were forced to wait and watch without going to medical institutions had a sudden change in their viral interstitial pneumonia condition and died, we hear reports that it was too late when the blood oxygen saturation started to fall.

Regarding COVID-19, among the PCR positive patients, in relation to the cases where some patients who had no or mild symptoms and were forced to wait and watch without going to medical institutions had a sudden change in their viral interstitial pneumonia condition and died, the present inventor realized that, to determine an appropriate treatment plan, it is important to understand “crepitations caused by interstitial pneumonia” exhibited before the blood oxygen saturation starts to fall, i.e., chronological changes in auscultatory sounds, such as a high-pitch “crackling [pa-ri-pa-ri]”auscultatory sound in a high frequency range, which is often in the range of −120 dB to −80 dB.

Accordingly, it is an object of the present invention to provide an auscultatory sound analysis system characterized by converting an auscultatory sound into digital data, further performing a spectrogram conversion, and outputting the strengths of a signal component at a specific frequency or in a specific frequency range along a time axis.

### Solution to Problem

To solve the problems described above, auscultatory sound analysis systems of the present invention include an auscultatory sound analysis system including:
(a) auscultatory sound signal acquisition means for acquiring an in-body auscultatory sound signal from the patient;
(b) auscultatory sound signal sampling means for digitally sampling and converting the in-body auscultatory sound signal into auscultatory sound discrete data; and
(c) spectrogram conversion means for converting the auscultatory sound discrete data into an auscultatory sound spectrogram, wherein
(d) on a basis of data acquired by the spectrogram conversion means, strengths of a signal component corresponding to at least one predetermined frequency are output along a time axis.

By using the auscultatory sound analysis system of the present invention over the course of time, it is possible to analyze clinical progression of the patient, by following the strengths of the signal component at the desired frequency along the time axis.

To solve the problems described above, the auscultatory sound analysis systems of the present invention include the auscultatory sound analysis system according to claim 1, wherein
(d) on the basis of the data acquired by the spectrogram conversion means, strengths of a signal component in at least one predetermined frequency range are output along a time axis.

By using the auscultatory sound analysis system of the present invention over the course of time, it is possible to analyze clinical progression of the patient, by following the strengths of the signal component in the desired frequency range along the time axis.

To solve the problems described above, the auscultatory sound analysis systems of the present invention include the auscultatory sound analysis system according to claim 1, wherein
(d) on the basis of the data acquired by the spectrogram conversion means, strengths of a signal component that exceed a prescribed threshold value in at least one predetermined frequency range are output along a time axis.

By using the auscultatory sound analysis system of the present invention over the course of time, it is possible to analyze clinical progression of the patient, by following, along the time axis, the strengths of the signal component that exceed the prescribed threshold value in the desired frequency range.

To solve the problems described above, among the auscultatory sound analysis systems of the present invention,
the auscultatory sound analysis system according to claim 4 makes it possible to visually recognize output data by using display means.

To solve the problems described above, the auscultatory sound analysis systems of the present invention include the auscultatory sound analysis system according to claim 1, 2, 3, or 4, incorporating:
a communication computation device provided with a display function, such as a smartphone.

By using the auscultatory sound analysis system of the present invention over the course of time, it is possible to display analysis information such as the strengths of the signal component by using a display screen of the smartphone. (FIG. 1B)

To solve the problems described above, the auscultatory sound analysis systems of the present invention include the auscultatory sound analysis system according to claim 1, 2, 3, 4, or 5, incorporating:
a body temperature thermometer and an electrocardiograph at a same time.

By using the auscultatory sound analysis system of the present invention over the course of time, it is possible to display information related to body temperatures and an electrocardiogram, together with the strengths of the signal component resulting from an auscultatory sound analysis. (FIG. 2)

To solve the problems described above, the auscultatory sound analysis systems of the present invention include the auscultatory sound analysis system according to claim 1, 2, 3, 4, 5, or 6, wherein
data generated in the auscultatory sound analysis system is uploaded into a cloud server on an Internet.

By using the auscultatory sound analysis system of the present invention over the course of time, it is possible to accumulate, in the cloud server, biological information acquired from the patient, together with the strengths of the signal component resulting from a spectrogram analysis of auscultatory sounds. (FIG. 1B)

To solve the problems described above, the auscultatory sound analysis systems of the present invention include the auscultatory sound analysis system according to claim 7, wherein
data related to an analysis is downloaded from a cloud server on the Internet.

By using the auscultatory sound analysis system of the present invention over the course of time, it is possible to acquire and to output or display judgment information after the biological information acquired from the patient is accumulated in the cloud server, together with the strengths of the signal component resulting from the spectrogram analysis of the auscultatory sounds. (FIG. 1B)

### Advantageous Effects of Invention

By using the auscultatory sound analysis system according to the present embodiments over the course of time, medical providers such as doctors and nurses are able to learn clinical conditions of patients that may change within a number of hours or a number of days, on the basis of changes in the auscultatory sound data, the body temperatures, and the electrocardiograms.

As shown in the example of the three-dimensional image display of a spectrogram of an auscultatory sound in [FIG. 5], on the assumption that energy is present in the same manner at 300 Hz and lower, the energy in the frequency band at 300 Hz and lower may be considered as meaningless as information used by a user for diagnosing respiratory sounds. It is therefore acceptable to make a correction so that the user is able to make diagnoses from various types of respiratory sounds on the basis of the energy in the frequency band at 300 Hz and higher, by making a correction to purposefully weaken lower frequency components. It is also understood that the low frequency zone at 300 Hz and lower has high energy, with respect to any of normal respiratory sounds, pneumonia respiratory sounds, and asthma respiratory sounds.

By using the auscultatory sound analysis system according to the present embodiment over the course of time, it is possible, in another medical application example, for a terminal to notify a medical provider of abnormalities such as when a waveform of a shunt sound in dialysis is different from normal or a frequency upper limit is exceeded. Further, it is also possible to provide a system capable of chronologically analyzing shunt auscultatory sounds during dialysis. In addition, needless to say, the present invention is also applicable to understanding clinical conditions of “drug-induced interstitial pneumonia”, which is a serious side effect of using medication such as an anti-cancer drug. It is possible to grasp and understand chronological changes in any disease subject to auscultation in clinical medicine, including those of respiratory organs, digestive organs, circulatory organs, and others.

### BRIEF DESCRIPTION OF DRAWINGS

FIG. 1A is a configuration diagram according to an embodiment of a device in an auscultatory sound analysis system of the present invention.

FIG. 1B is an auscultatory sound analysis system in an example where a smartphone is incorporated in a configuration.

FIG. 1C presents a schematic view of an Aurora Scope™ according to an embodiment example in which a device is pasted onto the chest of a patient.

FIG. 2 shows a GUI on a smartphone screen that is output wirelessly. The GUI displays, in a compact manner, a temperature (body temperature), a heartrate, a respiratory rate, a spectrogram of auscultatory sounds, and an electrocardiogram.

FIG. 3 shows a GUI on a smartphone screen that is output wirelessly. The GUI displays temperatures (body temperatures), heartrates, respiratory rates, and temporal changes in the “strengths of the signal components” in a plurality of desired frequency ranges of an auscultatory sound spectrogram, as well as change amounts in a desired part of the electrocardiogram.

FIG. 4 shows a GUI on a smartphone screen that is output wirelessly. The GUI displays temperatures (body temperatures), heartrates, respiratory rates, and temporal changes in the “strength of the signal component” in a desired frequency range of an auscultatory sound spectrogram.

FIG. 5 shows an example of three-dimensional image display of a spectrogram of an auscultatory sound.

On the assumption that energy is present in the same manner at 300 Hz and lower, the energy in the frequency band at 300 Hz and lower may be considered as meaningless as information used by a user for diagnosing respiratory sounds. It is therefore acceptable to make a correction so that the user is able to make diagnoses from various types of respiratory sounds on the basis of the energy in the frequency band at 300 Hz and higher, by making a correction to purposefully weaken lower frequency components. It is also understood that the low frequency zone at 300 Hz and lower has high energy, with respect to any of normal respiratory sounds, pneumonia respiratory sounds, and asthma respiratory sounds.

FIG. 6 presents a photograph of three-dimensional solid models of spectrograms of normal vesicular murmur (left) and crepitations from interstitial pneumonia (right) structured with green, yellow, red, and black Lego blocks. The frequency ranges are set to four zones, namely, 0 Hz to 500 Hz, 501 Hz to 1000 Hz, 1001 Hz to 1500 Hz, and 1500 Hz to 2000 Hz. For each of the zones, the status of the spectrogram is schematically expressed, the spectrograms each quantitatively analyzing an appearance status of the “strengths of the signal components”.

FIG. 7 presents a bird's eye view photograph of a three-dimensional solid model of a spectrogram of normal vesicular murmur structured with green and yellow Lego blocks. The frequency ranges are set to four zones, namely, 0 Hz to 500 Hz, 501 Hz to 1000 Hz, 1001 Hz to 1500 Hz, and 1500 Hz to 2000 Hz. For each of the zones, the status of the spectrogram is schematically expressed, the spectrogram quantitatively analyzing an appearance status of the “strengths of the signal components”.

FIG. 8 presents a bird's eye view photograph of a three-dimensional solid model of a spectrogram of crepitations caused by interstitial pneumonia structured with green, yellow, red, and black Lego blocks. The frequency ranges are set to four zones, namely, 0 Hz to 500 Hz, 501 Hz to 1000 Hz, 1001 Hz to 1500 Hz, and 1500 Hz to 2000 Hz. For each of the zones, the status of the spectrogram is schematically expressed, the spectrogram quantitatively analyzing an appearance status of the “strengths of the signal components”.

### DESCRIPTION OF EMBODIMENTS

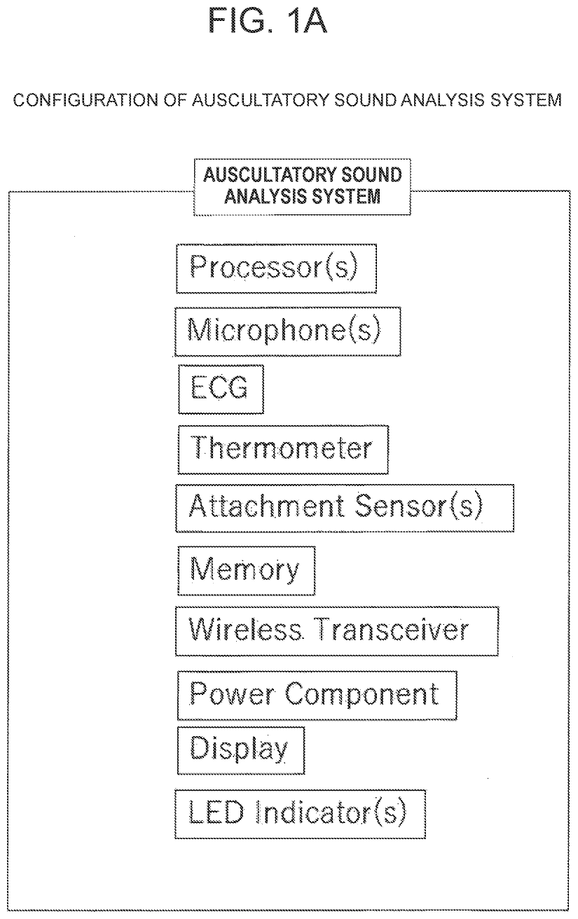

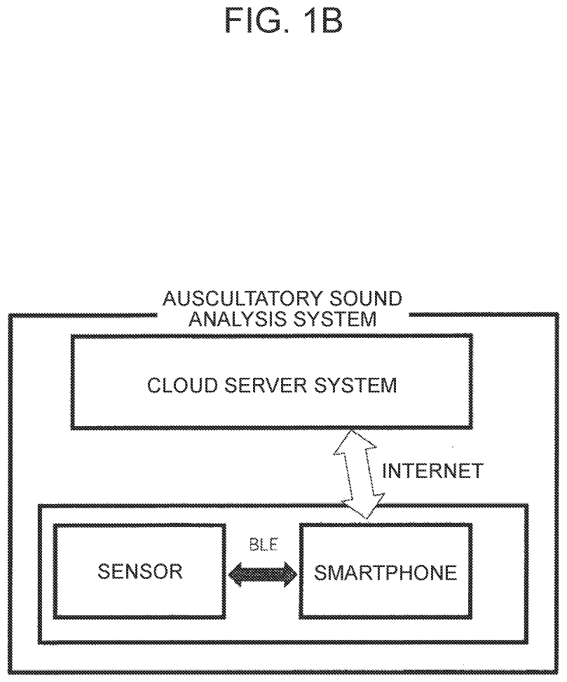

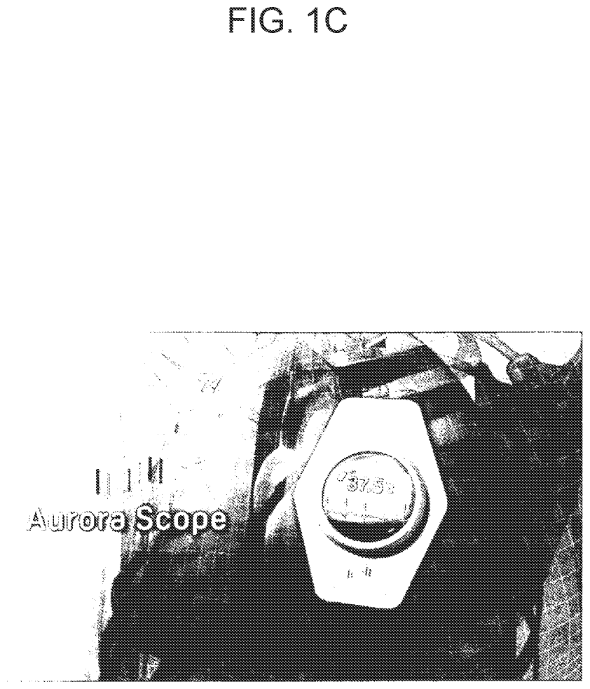

Next, embodiments of the present invention will be explained further in detail, with reference to the drawings.

[FIG. 1]A is a configuration diagram according to an embodiment of a device in an auscultatory sound analysis system of the present invention.

When contracted with COVID-19, one has fever at first, and subsequently has viral interstitial pneumonia, which causes an organ bloodstream failure due to blood coagulation in blood vessels 7 and causes, in particular, a malfunction of blood circulation in the heart. For this reason, the present invention adopts a configuration including a body temperature thermometer that makes it possible to understand an onset of the disease and a sensor having a device and a function that make it possible to understand changes in an electrocardiogram suggesting serious oxygen deficiency to myocardia. Also, an internal power supply is provided to enable independent electrical operations as a terminal device. In addition, a wireless communication unit is also provided for transmitting/receiving data to and from external devices. Examples of the microphone include a MEMS microphone and an organic/inorganic piezo microphone. Furthermore, a display unit for displaying data related to statuses of the patient is also provided.

[FIG. 1]B presents a system configuration diagram of an auscultatory sound analysis system of the present invention, showing an example in which a smartphone is included in the configuration. In this situation, it is possible to utilize functions of the smartphone for inputting, outputting, communication, computation, displaying, and billing.

[FIG. 1]C presents a schematic view of an Aurora Scope™ that carries out the system in an embodiment example.

The device is used as being pasted on the body surface at the chest of a patient.

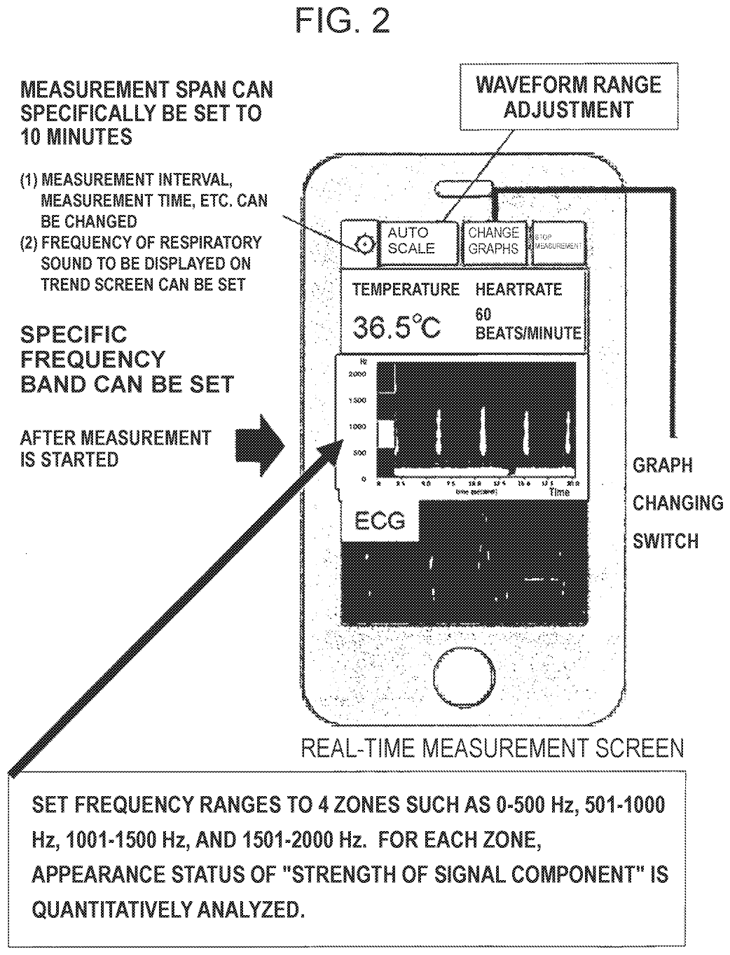

FIG. 2 shows a GUI on a smartphone screen that is output wirelessly. The GUI displays, in a compact manner, a temperature (body temperature), a heartrate, a respiratory rate, a spectrogram of auscultatory sounds, and an electrocardiogram. It is possible to easily understand the respiratory rate from appearance frequency of spectra in the spectrogram. In the present example, in the frequency range of 0 Hz to 500 Hz at the bottom of the spectrogram, the “strengths of the signal components” are constantly exhibited. In addition, near 1000 Hz, the “strengths of the signal components” are detected in conjunction with respiration. The present example shows the spectrogram based on the data from a case of drug-based interstitial pneumonia. By amplifying the amplitude of the auscultatory sounds, it is possible to “quantitatively” handle the “frequency range” corresponding to the particular disease.

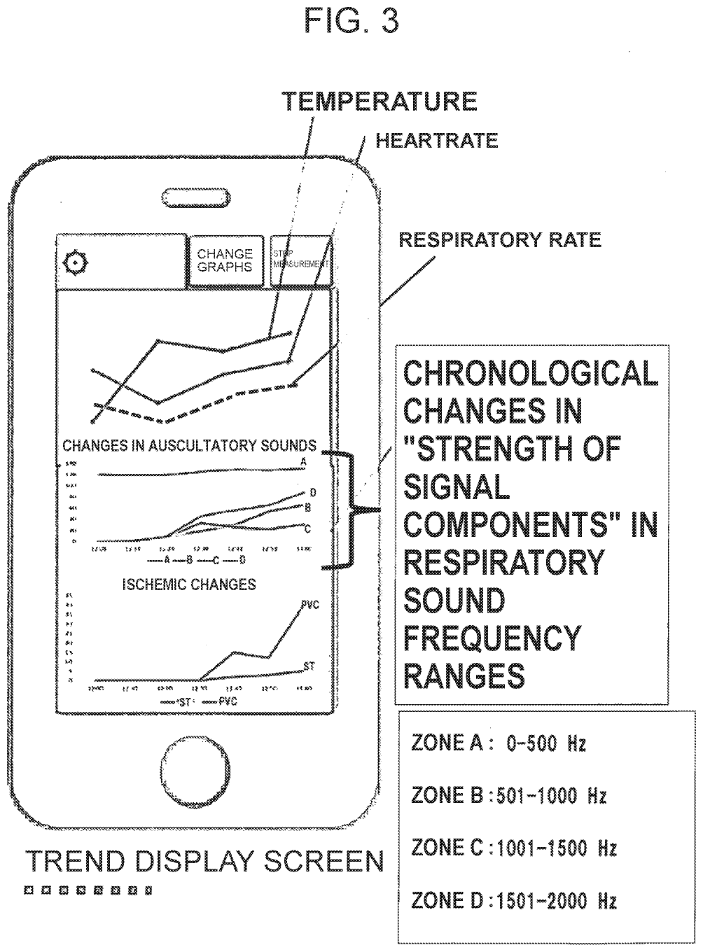

FIG. 3 shows a GUI on a smartphone screen that is output wirelessly. The GUI displays temperatures (body temperatures), heartrates, respiratory rates, and temporal changes in the “strengths of the signal components” in a plurality of desired frequency ranges of an auscultatory sound spectrogram, as well as change amounts in a desired part of the electrocardiogram, from the clinical condition described in paragraph [0042]. For an onset of respiration deficiency caused by COVID-19 which is of particular interest in the present disclosure, it is requisite to manage information in units of a number of days. Thus, it would be impossible, in the progression, to make judgments by looking at images of spectrograms of all the patients. Accordingly, this system allows us to understand chronological changes, on the notion that a change in the “strengths of the signal components” in a certain “frequency range” directly connected to a clinical condition within the spectrogram is directly connected to exacerbation of the clinical condition. As shown in the drawing, it is understood at a glance that the “respiratory rate” increases (start to huff and puff to breathe) as time passes and that, as for changes in the frequency range, the “strength of the signal component” increases in the crepitation zone representing a high-pitch “crackling [pa-ri-pa-ri]” sound.

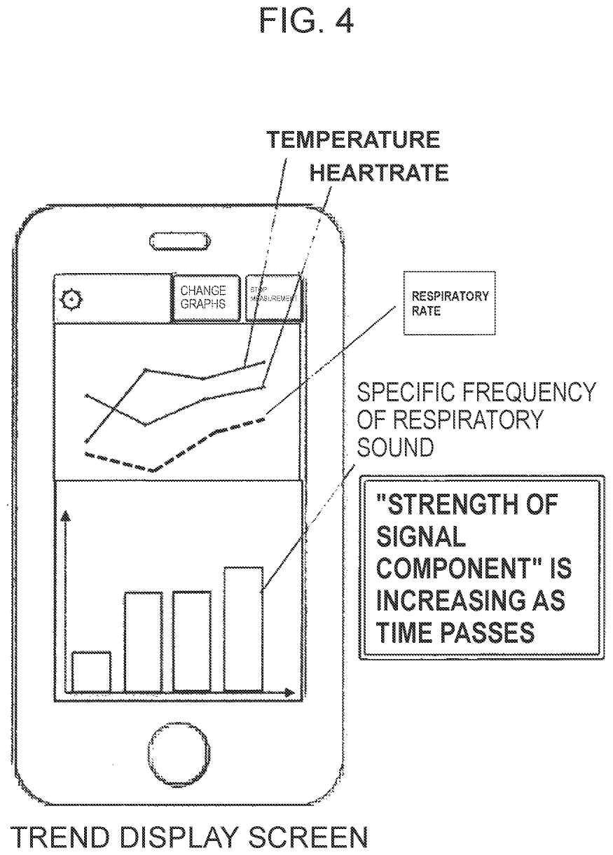

FIG. 4 shows a GUI on a smartphone screen that is output wirelessly. In addition to the description in paragraph [0043], the GUI displays temperatures (body temperatures), heartrates, respiratory rates, and temporal changes in the “strength of the signal component” in a desired frequency range of an auscultatory sound spectrogram. The present patent application is focused on high-pitch crepitations caused by interstitial pneumonia. However, as for respiratory auscultatory sounds, the capability to observe chronological progression of auscultatory sounds is useful in managing various clinical conditions, such as respiratory sounds from infantile asthma, and in the chest, noise from a valvular disease of the heart, as well as understanding from auscultatory sounds an artificial valve opening/closing malfunction soon after surgery, with regard to changes in opening/closing sounds of an artificial heart valve after the surgery on a valvular disease of the heart, detecting defects in a shunt sound from a dialysis shunt, and detecting auscultatory sounds of peristaltic movements of the abdominal intestines before diarrhea is caused by irritable bowel syndrome. In those situations, it is desirable to understand the chronological changes in the “strength of the signal component” by narrowing down the focus, in advance, on a frequency or a frequency range suitable for the specific purpose.

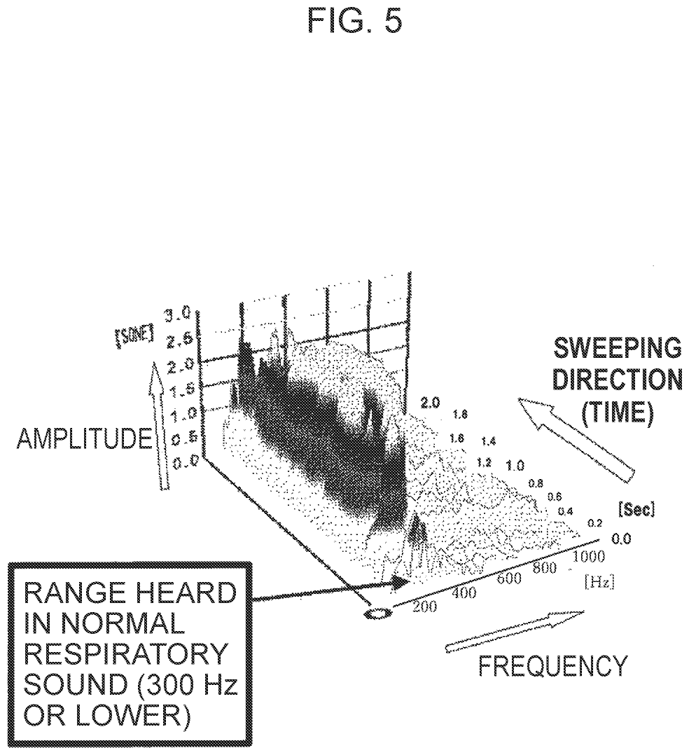

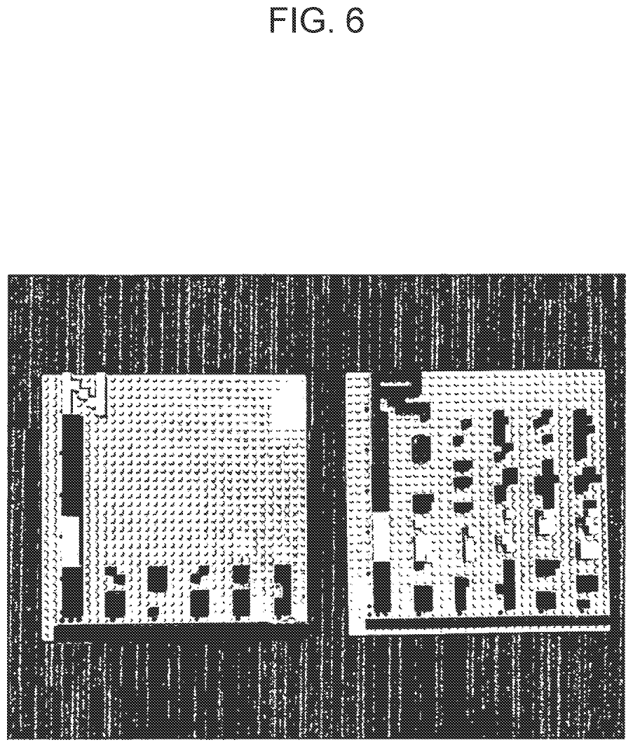

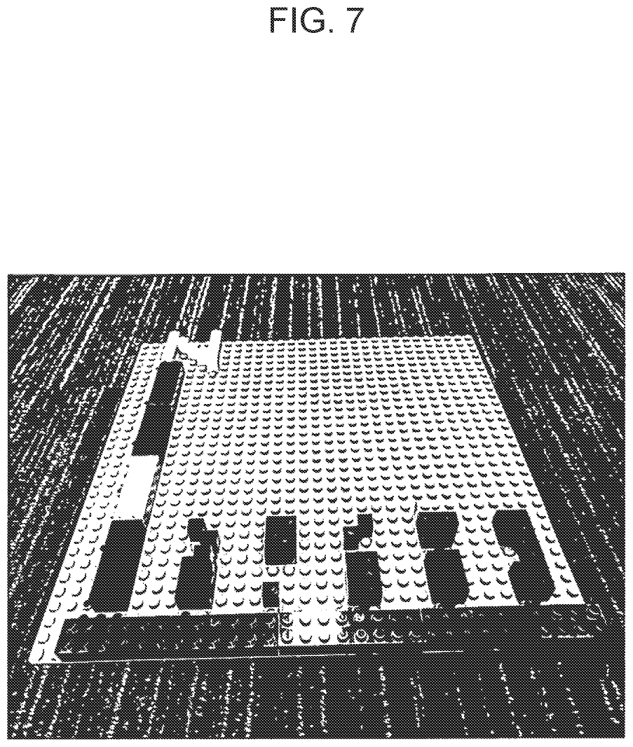

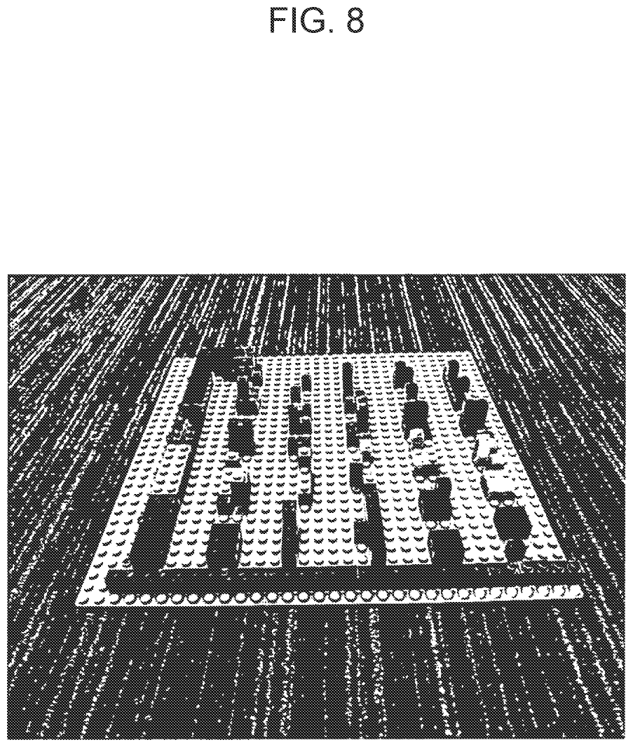

FIG. 5 shows an example of three-dimensional image display of a spectrogram of an auscultatory sound. On the assumption that energy is present in the same manner at 300 Hz and lower, the energy in the frequency band at 300 Hz and lower may be considered as meaningless as information used by a user for diagnosing respiratory sounds. It is therefore acceptable to make a correction so that the user is able to make diagnoses from various types of respiratory sounds on the basis of the energy in the frequency band at 300 Hz and higher, by making a correction to purposefully weaken lower frequency components. It is also understood that the low frequency zone at 300 Hz and lower has high energy, with respect to any of normal respiratory sounds, pneumonia respiratory sounds, and asthma respiratory sounds. Consequently, as set forth in claim 3, the present system is configured to extract the “strengths of the signal components” exceeding a prescribed threshold value so as to obtain the data thereof. It is therefore possible to acquire the data having less noise. Furthermore, setting the frequency range to the outside of the range of normal vesicular murmur makes it easier to understand abnormalities of the lungs.

FIG. 6 presents a photograph of three-dimensional solid models of spectrograms of normal vesicular murmur (left) and crepitations from interstitial pneumonia (right) structured with green, yellow, red, and black Lego blocks. The frequency ranges are set to four zones, namely, 0 Hz to 500 Hz, 501 Hz to 1000 Hz, 1001 Hz to 1500 Hz, and 1501 Hz to 2000 Hz. For each of the zones, the status of the spectrogram is schematically expressed, the spectrograms each quantitatively analyzing an appearance status of the “strengths of the signal components”. From this display, in order to understand a development status of the disease of COVID-19, it is suggested that a focus be placed on appearance frequency of the “strengths of the signal components” in the red and the black zones.

FIG. 7 presents a bird's eye view photograph of a three-dimensional solid model of a spectrogram of normal vesicular murmur structured with green and yellow Lego blocks. The frequency ranges are set to four zones, namely, 0 Hz to 500 Hz, 501 Hz to 1000 Hz, 1001 Hz to 1500 Hz, and 1501 Hz to 2000 Hz. For each of the zones, the status of the spectrogram is schematically expressed, the spectrogram quantitatively analyzing an appearance status of the “strengths of the signal components”. In this photograph, the magnitudes of the “strengths of the signal components” in the frequency range are expressed by using the bird's eye view. To cope with a premonitory phenomenon before exacerbation of interstitial pneumonia, a setting should be established so as to be able to pay attention to a signal appearing outside the zone at 500 Hz and lower corresponding to auscultatory sounds of normal vesicular murmur.

FIG. 8 presents a bird's eye view photograph of a three-dimensional solid model of a spectrogram of crepitations caused by interstitial pneumonia structured with green, yellow, red, and black Lego blocks. The frequency ranges are set to four zones, namely, 0 Hz to 500 Hz, 501 Hz to 1000 Hz, 1001 Hz to 1500 Hz, and 1501 Hz to 2000 Hz. For each of the zones, the status of the spectrogram is schematically expressed, the spectrogram quantitatively analyzing an appearance status of the “strengths of the signal components”.

In addition to the description in paragraph [0047], the appearance frequency and the magnitude of the “strength of the signal component” of the “pa” sound in the “crackling [pa-ri-pa-ri]” sound of the crepitations in a high-temperature zone specifically serve as a “sign” for development and exacerbation of clinical conditions. In order to be able to manage, through telemedicine, a patient of COVID-19 or the like convalescing without going to a medical institution, Bluetooth communication is used for the transmission/reception from a terminal module pasted on the patient's body surface to a smartphone, and further, the information is accumulated from the smartphone into a cloud server. In this manner, population health management is realized, which contributes to controlling clinical conditions in pandemics and the like and to medical welfare.

1-10. (canceled)

11. An auscultatory sound analysis system comprising:
(a) auscultatory sound signal acquisition means for acquiring an in-body auscultatory sound signal from a patient;
(b) auscultatory sound signal sampling means for digitally sampling and converting the in-body auscultatory sound signal into auscultatory sound discrete data; and
(c) spectrogram conversion means for converting the auscultatory sound discrete data into an auscultatory sound spectrogram, wherein
on a basis of data acquired by the spectrogram conversion means, strengths of a signal component in at least one predetermined frequency range are measured a plurality of times at intervals, the signal component exceeding a certain threshold value is extracted for each measurement, and the strengths of the signal component are output along a time axis.

12. The auscultatory sound analysis system according to claim 11, incorporating:
a communication computation device provided with a display function.

13. The auscultatory sound analysis system according to claim 11, incorporating:
a body temperature thermometer and an electrocardiograph.

14. The auscultatory sound analysis system according to claim 11, wherein
data generated in the auscultatory sound analysis system is uploaded into a cloud server on an Internet.

15. The auscultatory sound analysis system according to claim 14, wherein
data related to an analysis is downloaded from a cloud server on the Internet.

16. An auscultatory sound analysis system comprising:
(a) auscultatory sound signal acquisition means for acquiring an auscultatory sound signal;
(b) auscultatory sound signal sampling means for digitally sampling and converting the auscultatory sound signal into auscultatory sound discrete data; and
(c) spectrogram conversion means for converting the auscultatory sound discrete data into an auscultatory sound spectrogram, wherein
on a basis of data acquired by the spectrogram conversion means, strengths of a signal component in at least one predetermined frequency range are measured a plurality of times at intervals, the signal component exceeding a certain threshold value is extracted for each measurement, and the strengths of the signal component are output along a time axis.

17. The auscultatory sound analysis system according to claim 16, incorporating:
a communication computation device provided with a display function.

18. The auscultatory sound analysis system according to claim 16, wherein
data generated in the auscultatory sound analysis system is uploaded into a cloud server on an Internet.

19. The auscultatory sound analysis system according to claim 18, wherein
data related to an analysis is downloaded from a cloud server on the Internet.
